(19) United States
(12) Patent Application Publication
(10) Pub. No.: US 2026/0069537 A1
(43) Pub. Date: Mar. 12, 2026

(54) Stable Aqueous Bendamustine Cyclodextrin Composition

(71) Applicant: SoftKemo Pharma Corp., Montreal (CA)

(72) Inventor: Kishore Patel, Montreal (CA)

(21) Appl. No.: 19/321,353

(22) Filed: Sep. 8, 2025

Related U.S. Application Data
(60) Provisional application No. 63/732,657, filed on Sep. 6, 2024.

(51) Int. Cl. A61K 9/08; A61K 31/4184; A61K 47/02; A61K 47/26; A61K 47/40; A61K 47/69

(52) U.S. Cl. CPC A61K 9/08; A61K 31/4184; A61K 47/02; A61K 47/26; A61K 47/40; A61K 47/6951

(57) ABSTRACT

A pharmaceutical composition comprising: (a) bendamustine; (b) sulfobutyl ether beta-cyclodextrin (“SBECD”); (c) optionally, sodium chloride; and (d) aqueous diluent; characterized in that the SBECD concentration is between 40% and 60% (w/w) based upon the combined weight of the SBECD and aqueous diluent. In addition, a kit comprising (a) a vial of such liquid pharmaceutical composition and (b) a stabilizing agent; as well as a method of treating a cancer patient comprising administering to such patient an effective dose of such pharmaceutical composition.

### FIELD OF THE INVENTION

The present invention is directed to an aqueous pharmaceutical composition comprising (a) bendamustine; (b) sulfobutyl ether beta-cyclodextrin (“SBECD”); (c) optionally sodium chloride; and (d) an aqueous diluent, characterized in that the SBECD concentration is between 40% and 60% (w/w) based upon the combined weight of the SBECD and aqueous diluent. Such composition exhibits desirable storage stability when stored at refrigerated temperatures (for example of between 2° and 8° C.) without being lyophilized. In another aspect this invention is directed to a kit comprising (a) a vial of such liquid pharmaceutical composition and (b) a stabilizing agent. In a third aspect, this application is directed to a method of treating a cancer patient comprising administering to such patient an effective dose of such pharmaceutical composition which has been stored in liquid form without being lyophilized prior to such administration.

### BACKGROUND OF THE INVENTION

Bendamustine, 4-[5-[Bis(2-chloroethyl)amino]-1-methylbenzimidazol-2-yl]butanoic acid, is a nitrogen mustard compound which has been found to be useful in the treatment of leukemia and lymphomas, particularly Chronic Lymphocytic Leukemia (CLL) and Indolent Non-Hodgkin Lymphoma (iNHL). Further, as is disclosed in U.S. Pat. No. 8,703,964 (Popek et al), bendamustine in complex form has been shown to be effective against solid tumors, including lung cancer, breast cancer and multiple myeloma.

Unfortunately, bendamustine is highly reactive with water and therefore commercial formulations of bendamustine are often lyophilized so that they can be stored for longer periods of time. For example, U.S. Pat. No. 8,791,270 (Brittain et al) specifically teaches that, due to its degradation in aqueous solutions, bendamustine is supplied as a lyophilized product. Similarly, publications specifically directed to bendamustine compositions comprising SBECD indicate that such formulations need to be lyophilized in order to exhibit long-term stability; see US Patent Application 2024/0148696 (Patel et al) and US Patent Application 2022/0304983 (Jing et al).

Liquid compositions of bendamustine, described in U.S. Pat. No. 8,609,707 (Palepu et al), have been commercialized (Bendeka® and Belrapzo®). These formulations possess an advantage over lyophilized compositions in that the need for reconstitution of the lyophilized formulation (which requires up to 5 minutes of shaking for the commercial lyophilized product Treanda®) is avoided. However, these formulations comprise substantial amounts of organic solvents, specifically a combination of polyethylene glycol and propylene glycol, as well as of monothioglycerol (a stabilizer), all of which need to be metabolized thereby creating an additional burden on weakened cancer patients.

It has now been unexpectedly found that a pharmaceutical composition comprising ((a) (a) bendamustine; (b) sulfobutyl ether beta-cyclodextrin (“SBECD”); (c) optionally sodium chloride; and (d) an aqueous diluent, characterized in that the SBECD concentration is between 40% and 60% (w/w) based upon the combined weight of the SBECD and aqueous diluent; exhibits desirable storage stability when stored at refrigerated temperatures (e.g., between 2° and 8° C.) without the need of being lyophilized and then reconstituted. Further, such liquid compositions do not require the presence of organic solvent(s) to achieve desirable stability.

### SUMMARY OF THE INVENTION

In one aspect, the present invention is directed to a pharmaceutical composition comprising:
(a) bendamustine;
(b) sulfobutyl ether beta-cyclodextrin (“SBECD”);
(c) optionally, sodium chloride; and
(d) an aqueous diluent;
characterized in that the SBECD concentration is between 40% and 60% (w/w) based upon the combined weight of the SBECD and aqueous diluent.

In another aspect, this invention is directed to a kit comprising:
(i) a vial of a liquid pharmaceutical composition comprising:
   (a) bendamustine;
   (b) sulfobutyl ether beta-cyclodextrin (“SBECD”);
   (c) optionally, sodium chloride; and
   (d) an aqueous diluent;
characterized in that the SBECD concentration is between 40% and 60% (w/w) based upon the combined weight of the SBECD and aqueous diluent; and
   (ii) a cationic stabilizing agent.

In yet another aspect this invention is directed to a method for treating a cancer patient comprising administering to such patient a therapeutically effective dose of a composition produced by diluting a pharmaceutical composition comprising (a) bendamustine; (b) sulfobutyl ether beta-cyclodextrin (“SBECD”); (c) optionally, sodium chloride; and (d) aqueous diluent; characterized in that the SBECD concentration is between 40% and 60% (w/w) based upon the combined weight of the SBECD and aqueous diluent; which has been stored in liquid form (i.e., without being lyophilized) prior to such administration . . .

### DETAILED DESCRIPTION OF THE INVENTION

In one aspect, the present invention is directed to a pharmaceutical composition comprising:
(a) bendamustine;
(b) sulfobutyl ether beta-cyclodextrin (“SBECD”);
(c) optionally, sodium chloride; and
(d) an aqueous diluent;
characterized in that the SBECD concentration is between 40% and 60% (w/w) based upon the combined weight of the SBECD and aqueous diluent.

As is employed herein, the term “pharmaceutical composition”, unless the context implies otherwise, means a composition that is made under conditions such that it is suitable for administration to humans, e.g., it is made under GMP conditions and contains (if present) pharmaceutically acceptable excipients.

As is employed herein, the term “bendamustine”, unless the context implies otherwise, means a bendamustine moiety in complex form with SBECD, produced when an appropriate bendamustine precursor compound is mixed with SBECD in an aqueous environment.

The term “bendamustine precursor compound” means a compound which will form a bendamustine moiety capable of forming an inclusion complex with SBECD when dissolved in an aqueous environment. Preferred bendamustine precursor compounds include bendamustine hydrochloride and bendamustine hydrochloride monohydrate.

The term “aqueous diluent” means any water based diluent which is suitable for pharmaceutical use. Preferred aqueous diluents include 0.9% Sodium Chloride Injection; 2.5% Dextrose/0.45% Sodium Chloride Injection; 5% Dextrose Injection and water for injection.

As is employed herein, the term “therapeutically effective dose”, unless the context implies otherwise, as used herein refers to that amount of the compound being administered that will relieve to some extent one or more of the symptoms of the disorder being treated. In reference to the treatment of neoplasms, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth, and/or, (4) relieving to some extent (or, preferably, eliminating) one or more symptoms associated with the cancer.

The pharmaceutical compositions of this invention may be prepared by the dissolution of a solid bendamustine precursor, such as bendamustine hydrochloride and/or bendamustine hydrochloride monohydrate in an aqueous solution of SBECD; or by mixing an aqueous solution of SBECD with an aqueous stock solution of bendamustine. Sodium chloride may be added to the aqueous solution of SBECD either before or after the addition of the bendamustine precursor; or alternatively, to the stock solution of bendamustine either before or after the addition of SBECD. Such aqueous solutions of bendamustine or SBECD used to prepare the compositions of this invention may comprise water for injection or another pharmaceutically acceptable water based diluent. In one preferred embodiment, the pharmaceutical composition is prepared by adding bendamustine and SBECD to an aqueous sodium chloride solution (such as saline; or saline to which additional sodium chloride has been added).

In certain embodiments, the compositions of this invention may be prepared by (a) first forming a lyophilized composition of bendamustine and SBECD, using processes such as those described in US Patent Application 2024/0148696 (Patel et al) or the like; and (b) then diluting such lyophilized with a sufficient amount of an acceptable aqueous diluent such that the SBECD concentration is between 40% and 60% (w/w) based upon the combined weight of the SBECD and water.

When bendamustine is combined with SBECD is an aqueous environment, the compounds form an inclusion complex in a 1:1 molar ratio. In forming such inclusion complex, the bendamustine moiety interacts with and becomes sheltered within the cavity of the torus-like structure formed by the cyclic cyclodextrin units of SBECD, thereby sheltering the bendamustine moiety from the degrading effects of an aqueous environment. In order to provide a composition which is amenable to rapid administration to cancer patients, it is desirable to ensure that the bendamustine moiety is not fully saturated in the reconstituted solution at room temperature. Accordingly, the compositions of this invention typically comprise a molar excess of SBECD, which ensures that all of the bendamustine precursor will remain in complex form (and will not precipitate) during the infusion process. Preferably, the molar ratio of bendamustine to SBECD is between about 1:2 and about 1:25; is more preferably between about 1:3 and about 1:15; is even more preferably between about 1:4 and about 1:10 and most preferably between about 1:5 and about 1:7.

SBECD may be present in an amount between 40% and 60% (w/w), based upon the amount of SBECD and aqueous diluent present. Preferably, SBECD is present in an amount between 40% and 50% (w/w) as such compositions are more amenable to being filtered.

Bendamustine may be present in an amount sufficient to comply with the molar bendamustine:SBECD ratios discussed above, which amounts may readily be determined by one of ordinary skill in the art. Preferably, bendamustine is added in an amount such that a 25 mL vial will contain 90.76 mg of bendamustine moiety in complex form [an amount equivalent to the amount of bendamustine moiety present in 100 mg of bendamustine hydrochloride]. Thus, for example, the following formulations may be preferably employed:

| SBECD in | SBECD | Bendamustine | Water |
|---|---|---|---|
| Formulation (w/w) | (g) | Hydrochloride (mg) | (g) |
| 40% | 3.33 | 100 | 5.00 |
| 50% | 3.33 | 100 | 3.33 |
| 60% | 3.33 | 100 | 2.22 |

As is demonstrated in the examples below, it has been found that the addition of sodium chloride to the composition of this invention will enhance its storage stability under refrigerated conditions (typically at temperature of 2-8° C.). In general, sodium chloride may be added in amounts up to about 10% (w/w) based upon the amount of water present. Such enhanced stability increases in accordance with the concentration of sodium chloride added; however, it is generally preferred to employ lower concentrations (or none) in order to be able to treat patients who require a lower sodium intake. Consequently, in general, it is preferred to employ NaCl in amounts ranging from 0% to 1.2% (w/w) based upon the amount of water present in the composition. In those formulations in which sodium chloride is to be present, it is typically most convenient to use saline (having a 0.9% w/w concentration) as the solvent for SBECD and the bendamustine precursor employed.

In another aspect, this invention is directed to a kit comprising:
(i) a vial of a liquid pharmaceutical composition comprising:
   (a) bendamustine;
   (b) sulfobutyl ether beta-cyclodextrin (“SBECD”);
   (c) optionally, sodium chloride; and
   (d) an aqueous diluent;
characterized in that the SBECD concentration is between 40% and 60% (w/w) based upon the combined weight of the SBECD and aqueous diluent; and
(ii) a cationic stabilizing agent.

In such kit, the stabilizing agent is packaged independently of the liquid pharmaceutical composition, in a manner which will enable such agent to remain stable when stored at refrigerated temperature (e.g., of 2°−8° C.) for a period of time at least equivalent to the storage capacity of the liquid pharmaceutical composition. In this regard, the storage capacity of the liquid pharmaceutical composition refers to the amount of time that such composition will maintain a bendamustine concentration of at least 95% when stored at 2°−8° C.

The cationic stabilizing agents employed in such kits include those described in U.S. Pat. No. 8,436,032 (Popek et al) and U.S. Pat. No. 8,383,623 (Alakhov et al); as well as those described in US Patent Application 2022/0304983 (Jing et al). Cationic agents which may be employed include tertiary or quaternary ammonium compounds, such as N-alkyl-N,N-dimethylamines, N-alkyl-N,N-diethylamines, N-alkyl-N—N-diethanoloamines, N-alkylmorpholine, N-alkylpiperidine, N-alkylpyrrolidine, N-alkyl-N,N,N-trimethylammonium, N,N-dialkyl-N,N-dimethylammonium, N-alkyl-N-benzyl-NN-diimethylammonium, N-alkyl-pyridinium, N-alkyl-picolinium, alkylamidomethylpyridinium, carbalkoxypyridinium, N-alkylquinolinium, N-alkylisoquinolinium, N,N-alkylmethylpyrollidinium, and 1-alkyl-2,3-dimethylimidazolium. Particularly preferred cationic adjuvants include sterically hindered tertiary amines, such as N-alkyl-N—N-diisopropylamine, N-alkylmorpholine, N-alkylpiperidine, and N-alkylpyrrolidine; and quaternary ammonium compounds such as cetylpyridinium chloride, benzyldimethyldodecylammonium chloride, dodecylpyridinium chloride, hexadecyltrimethylammonium chloride, benzyldimethyltetradecylammonium chloride, octedecyldimethylbenzylammonium chloride, and domiphen bromide.

Polycationic compounds such as oligo- or polyamines, or pegylated oligo- or polyamines may also be employed as the stabilizing agent. Preferred polycationic compounds include oligoamines such as spermin, spermidin, putrescine, and cadaverine; polyamines: such as polyethyleneimine, polyspermin, polyputrescine, and polycadaverine; and pegylated oligoamines and polyamines of the group listed above. Particularly preferred is PI2080, polyethyleneimine 2000 conjugated with PEG 8000.

One preferred class of cationic stabilizing agents are polypeptides comprising from about 5 to about 50, more preferably between about 6 and about 20, amino acids; wherein at least about 50% of such amino acids contain a positive charge. Most preferably, such charged amino acids are argenine. Particularly preferred members of this class include polyargenine and protamine which has been digested with thermolysin (hereinafter referred to as Low Molecular Weight Protamine or “LMWP”).

Hydrophobically modified oligo- or polyamines may also be employed. Preferred stabilizing agent of this type include acetyl spermin, acetyl polyspermin, acetyl polyethyleneimine, butyryl spermin, butyryl polyspermin, butyryl polyethyleneimine, lauroyl spermin, lauroyl polyspermin, lauroyl polyethyleneimine, stearoyl spermin, stearoyl polyspermin, and stearoyl polyethyleneimine,

In addition, cationic polysaccharides and synthetic polycationic polymers may also be employed. Illustrative of such cationic polysaccharides are chitosan, deacetylated chitosan, quaternized cellulose, quaternized amylase, quaternized amylopectine, quaternized partially hydrolyzed cellulose, quaternized partially hydrolyzed amylase and quaternized partially hydrolyzed amylopectine. Illustrative of such synthetic polycationic polymers are polyquaternium 2 (poly[bis(2-chloroethyl]ether-alt-1,3-bis[3-dimethylamino) propyl]-urea, quaternized); polyquaternium 11 (poly(1-vinylpyrrolidone-co-dimethylammonioethyl methacrylate) quaternized); polyquaternium 16 and 44 (copolymer of vinylpyrrolidone, and quaternized vinylimidazole); and polyquaternium 46 (copolymer of vinylcaprolactam, vinylpyrrolidone, and quaternized vinylimidazole).

Although the cationic cyclopolysaccharides which may be employed as stabilizing agents may comprise any one or mixture of cationic groups, in general it is preferred that such compound comprise an amino, a guanidine or a quaternary ammonium group. Illustrative of the amino-cyclodextrins which may be preferably employed include are amino-alpha-cyclodextrincyclodextrins, amino-beta-cyclodextrincyclodextrins, and amino-gamma-cyclodextrin, each cyclodextrins, preferably having a substitution level of between about 4 and about 10. Preferred amino-cyclodextrins of this type include hexakis (6-amino-6-deoxy) alpha-cyclodextrin, heptakis (6-amino-6-deoxy) beta-cyclodextrin, octakis (6-amino-6-deoxy) gamma-cyclodextrin and heptakis (6-amino-6-deoxy) beta-cyclodextrin. Other cationic cyclopolysaccharides which may be employed including guanidino-cyclodextrins, preferably having a substitution level of between about 4 and about 10, with heptakis (6-guanidino-6-deoxy) beta-cyclodextrin being particularly preferred; and alkylamino-cyclodextrins, preferably having a substitution level of between about 4 and about 10, with as 6-deoxy-6-(3-hydroxy) propylamino beta-cyclodextrin being particularly preferred.; and ammonium-cyclodextrins, preferably having a substitution level between 4 and 9, such as 2-hydroxy-N,N,N-trimethylpropanammonium-cyclodextrin.

Particularly preferred cationic polysaccharides include hexakis (6-amino-6-deoxy) alpha-cyclodextrin, heptakis (6-amino-6-deoxy) beta-cyclodextrin, octakis (6-amino-6-deoxy) gamma-cyclodextrin, heptakis (6-guanidino-6-deoxy) beta-cyclodextrin, octakis (6-guanidino-6-deoxy)-gamma-cyclodextrin, 2-hydroxy-N,N,N-trimethylpropanammonium-cyclodextrin and 6-deoxy-6-(3-hydroxy) propylamino beta-cyclodextrin; with 6-deoxy-6-(3-hydroxy) propylamino beta-cyclodextrin being especially preferred.

The cationic stabilizing agent is typically provided in a molar ratio of between about 5:1 and about 1:1000; preferably of between about 1:4 and about 1:100, based upon the molar amount of SBECD present in the vial containing the liquid pharmaceutical composition.

In yet another aspect this invention is directed to a method for treating a cancer patient comprising administering to such patient a therapeutically effective dose of a composition comprising (a) bendamustine; (b) sulfobutyl ether beta-cyclodextrin (“SBECD”); (c) optionally, sodium chloride; and (d) an aqueous diluent; characterized in that the SBECD concentration is between 40% and 60% (w/w) based upon the combined weight of the SBECD and aqueous diluent.

In certain preferred embodiments, prior to administration to a patient, a measured sample of the pharmaceutical composition described above is diluted with a pharmaceutically acceptable diluent until the desired concentration of bendamustine in such dosing solution is achieved. Preferred diluents include 0.9% Sodium Chloride Injection; 2.5% Dextrose/0.45% Sodium Chloride Injection; 5% Dextrose Injection and water for injection. In the event that a cationic stabilizing agent is present, such agent may be blended into the dosing solution prior to administration.

The recommended dosage for CLL is 100 mg/m² of bendamustine hydrochloride administered intravenously over 10 minutes on Days 1 and 2 of a 28-day cycle, up to 6 cycles. The recommended dosage for iNHL is 120 mg/m² of bendamustine hydrochloride administered intravenously over 10 minutes on Days 1 and 2 of a 21-day cycle, up to 8 cycles.

The amount of reconstituted solution necessary to achieve the required dose is then typically added to a 150 mL partial additive bag (PAB) containing 50 mL of the same diluent that was used for reconstitution. Prior to administration, the PAB contents are mixed by gently inverting the bag back and forth 20 times.

For CLL indication the volume of the dosing solution in the PAB ranges from 60 mL for a 1 m² patient to 80 mL for a 3 m² patient. and the corresponding bendamustine concentrations range from 1.51 mg/mL for a 1 m² patient to 3.4 mg/mL for a 3 m² patient; which are equivalent to 1.67 mg/mL to 3.75 mg/mL of bendamustine hydrochloride, respectively.

For iNHL indication the admixture ranges from 62 mL for a 1 m² patient to 86 mL for a 3 m² patient and the corresponding bendamustine concentrations range from 1.76 mg/mL for a 1 m² patient to 3.80 mg/mL for a 3 m² patient; which are equivalent to 1.94 mg/mL to 4.19 mg/mL of bendamustine hydrochloride, respectively.

Because such diluted formulations are not fully saturated, the compositions may be administered in less than about 15 minutes, preferably in less than about 10 minutes.

In other embodiments, the liquid formulations of this invention may be administered in the form of implants, patches or suppositories; as well as by injection or infusion. Such administration may involve further diluting the liquid formulation with an acceptable aqueous diluent prior to administration.

It is to be understood that each component, compound, substituent, or parameter disclosed herein is to be interpreted as being disclosed for use alone or in combination with one or more of each and every other component, compound, substituent, or parameter disclosed herein.

It is also to be understood that each amount/value or range of amounts/values for each component, compound, substituent, or parameter disclosed herein is to be interpreted as also being disclosed in combination with each amount/value or range of amounts/values disclosed for any other component(s), compounds(s), substituent(s), or parameter(s) disclosed herein and that any combination of amounts/values or ranges of amounts/values for two or more component(s), compounds(s), substituent(s), or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, substituent, or parameter disclosed in the description or an example is to be interpreted as a disclosure of either a lower or an upper limit of a range and thus can be combined with any other lower or upper limit of a range or specific amount/value for the same component, compound, substituent, or parameter disclosed elsewhere in the application to form a range for that component, compound, substituent, or parameter.

### EXAMPLES

The following examples are provided to illustrate the invention in accordance with the principles of this invention, but are not to be construed as limiting the invention in any way except as indicated in the appended claims.

### Examples 1-6: Stability of Bendamustine/SBECD Complex Compositions Stored at 5° C.

### Example 1

1000 mg of bendamustine hydrochloride and 33.33 g of sodium sulfobutyl ether β-cyclodextrin were dissolved in 48.98 g of water for injection to provide a clear solution. This clear solution was filtered through a 0.22 μm filter and aliquoted into glass vials. Each vial contained the equivalent of 10 mg of bendamustine hydrochloride in a 40% solution (w/w) of sodium sulfobutyl ether β-cyclodextrin in water for injection.

### Example 2

1000 mg of bendamustine hydrochloride and 33.33 g of sodium sulfobutyl ether β-cyclodextrin were dissolved in 48.98 g of 0.9% NaCl to provide a clear solution. This clear solution was filtered through a 0.22 μm filter and aliquoted into glass vials. Each vial contained the equivalent of 10 mg of bendamustine hydrochloride in a 40% solution (w/w) of sodium sulfobutyl ether β-cyclodextrin in 0.9% NaCl.

### Example 3

1000 mg of bendamustine hydrochloride and 33.33 g of sodium sulfobutyl ether β-cyclodextrin were dissolved in 32.33 g of water for injection (WFI) to provide a clear solution. This clear solution was filtered through a 0.22 μm filter and aliquoted into glass vials. Each vial contained the equivalent of 10 mg of bendamustine hydrochloride in a 50% solution (w/w) of sodium sulfobutyl ether β-cyclodextrin in water for injection.

### Example 4

1000 mg of bendamustine hydrochloride and 33.33 g of sodium sulfobutyl ether β-cyclodextrin were dissolved in 32.33 g of 0.9% NaCl to provide a clear solution. This clear solution was filtered through a 0.22 μm filter and aliquoted into glass vials. Each vial contained the equivalent of 10 mg of bendamustine hydrochloride in a 50% (w/w) solution of sodium sulfobutyl ether β-cyclodextrin in 0.9% NaCl.

### Example 5

1000 mg of bendamustine hydrochloride and 33.33 g of sodium sulfobutyl ether β-cyclodextrin were dissolved in 21.22 g of water for injection to give a clear solution. This clear solution was filtered through a 0.22 μm filter and aliquoted into glass vials. Each vial contained the equivalent of 10 mg of bendamustine hydrochloride in a 60% solution (w/w) of sodium sulfobutyl ether β-cyclodextrin in water for injection.

### Example 6

1000 mg of bendamustine hydrochloride and 33.33 g of sodium sulfobutyl ether β-cyclodextrin were dissolved in 21.22 g of 0.9% NaCl to give a clear solution. This clear solution was filtered through a 0.22 μm filter and aliquoted in glass vials. Each vial contained the equivalent of 10 mg of bendamustine hydrochloride in a 60% solution (w/w) of sodium sulfobutyl ether β-cyclodextrin in 0.9% NaCl.

The samples of Examples 1-6 were sparged with N₂, and sealed. The samples were maintained at 2-5° C. and analyzed for their initial bendamustine content as well as after 1, 2, 3 and 5 months (by HPLC). The area under the curve (AUC), employing bendamustine hydrochloride as a reference, was used to evaluate the extent of bendamustine degradation. The results, expressed as % remaining bendamustine, are summarized in Table 1 below.

TABLE 1
Stability at 5° C

| Example | 1 | 2 | 3 | 4 | 5 | 6 |
|---|---|---|---|---|---|---|
| SBECD | 40% | 40% | 50% | 50% | 60% | 60% |
| (w/w) | | | | | | |
| Diluent | WFI | 0.9% NaCl | WFI | 0.9% NaCl | WFI | 0.9% NaCl |
| Initial | 99.16 | 99.76 | 99.44 | 99.67 | 99.55 | 99.67 |
| 1 month | 98.42 | 99.41 | 98.88 | 99.44 | 99.25 | 99.57 |
| 2 months | 97.59 | 99.02 | 98.39 | 99.22 | 99.03 | 99.47 |
| 3 months | 96.54 | 98.65 | 97.97 | 99.01 | 98.83 | 99.33 |
| 5 months | 95.56 | 98.25 | 97.27 | 98.67 | 98.58 | 99.12 |

The above results indicate that the liquid formulations of this invention exhibit desirable storage stability; and that such stability is enhanced when saline is employed in place of water for injection.

### Examples 7-15: Accelerated Stability Testing of Bendamustine/SBECD Complex Formulations at 42° C.

Formulations of bendamustine in an inclusion complex with SBECD were prepared by dissolving bendamustine hydrochloride in 50% w/w solution of sodium sulfobutyl ether β-cyclodextrin (SBECD) in water for injection (WFI). The formulations were preincubated at 10° C. for 15 minutes on ultrasonic bath; and then mixed with sodium chloride in the amounts indicated in Table 2 below. The formulations were incubated in an ultrasonic bath for 30 minutes at 10° C. and filtered through 0.22 μm filter. The final concentration of bendamustine in all formulations was 10 mg/g (based upon the weight of WFI).

The formulations were incubated at 42° C. for 12 days. Aliquots were diluted to a bendmustine concentration of 0.5 mg/g using 0.9% NaCl and were analyzed by HPLC. The area under the curve (AUC) was used to evaluate percentage of bendamustine remaining (using bendamustine hydrochloride as the standard).

The results of such analysis are presented in Table 2 below.

TABLE 2
Bendamustine Assay After Storage at 42° C. for 12 Days

| | NaCl Added | Bendamustine |
|---|---|---|
| Example | (mg/g WFI) | Percent Remaining |
| 7 | 0 | 49.4 |
| 8 | 3 | 49.9 |
| 9 | 6 | 53.0 |
| 10 | 9 | 61.1 |
| 11 | 12 | 67.1 |
| 12 | 15 | 71.6 |
| 13 | 22 | 76.7 |
| 14 | 30 | 77.1 |
| 15 | 80 | 92.4 |

The above data shows that the stability of the formulation increases with the amount of NaCl added.

What is claimed is:

1. A pharmaceutical composition comprising:
(a) bendamustine;
(b) sulfobutyl ether beta-cyclodextrin (“SBECD”);
(c) optionally, sodium chloride; and
(d) an aqueous diluent;
characterized in that the SBECD concentration is between 40% and 60% (w/w) based upon the combined weight of the SBECD and aqueous diluent.

2. The pharmaceutical composition of claim 1 wherein the molar ratio of bendamustine to SBECD is between 1:3 and 1:15.

3. The pharmaceutical composition of claim 2 wherein the molar ratio of bendamustine to SBECD is between 1:4 and 1:10.

4. The pharmaceutical composition of claim 3 wherein the molar ratio of bendamustine to SBECD is between 1:5 and 1:7.

5. The pharmaceutical composition of claim 1 wherein the SBECD concentration is between 40% and 50% (w/w) based upon the combined weight of the SBECD and aqueous diluent.

6. The pharmaceutical composition of claim 1 wherein sodium chloride is present in an amount between 0% and 1.2% (w/w) based upon the amount of aqueous diluent present in the composition.

7. A kit comprising:
(i) a vial containing the pharmaceutical compositions of claim 1; and
(ii) a cationic stabilizing agent.

8. The kit of claim 7 wherein the stabilizing agent is 6-deoxy-6-(3-hydroxy) propylamino beta-cyclodextrin.

9. A method of treating a cancer patient comprising administering to said patient a therapeutically effective dose of the composition of claim 1.

10. The method of claim 9 wherein such administration is achieved by injection, infusion or via the use of a patch, implant or suppository.

11. The method of claim 9 comprising administering to such patient a therapeutically effective dose of a composition produced by diluting the pharmaceutical composition with a pharmaceutically acceptable diluent.

12. The method of claim 11 wherein the pharmaceutically acceptable diluent is selected from the group consisting of 0.9% sodium chloride injection; 2.5% dextrose/0.45% sodium chloride injection; 5% dextrose injection; and water for injection.
